(19) United States
(12) Patent Application Publication
(10) Pub. No.: US 2003/0026788 A1
(43) Pub. Date: Feb. 6, 2003

(54) Use of extracellular matrix tissue to preserve cultured cell phenotype

(76) Inventor: Bret A. Ferree, Cincinnati, OH (US)

Correspondence Address: John G. Posa, Gifford, Krass, Groh, Sprinkle,, Anderson & Citkowski, PC, 280 N. Old Woodward Ave., Suite 400, Birmingham, MI, 48009, US

(21) Appl. No.: 10/253,211

(22) Filed: Sep. 24, 2002

Related U.S. Application Data
(63) Continuation-in-part of application No. 09/688,716, filed on Oct. 16, 2000, now Pat. No. 6,454,804.
Application No. 09/688,716 is a continuation-in-part of application No. 09/638,726, filed on Aug. 14, 2000, now Pat. No. 6,340,369.
Application No. 09/688,716 is a continuation-in-part of application No. 09/415,382, filed on Oct. 8, 1999, now Pat. No. 6,419,704.

(51) Int. Cl. A61K 45/00; C12N 5/08

(52) U.S. Cl. 424/93.7; 435/366

(57) ABSTRACT

Pieces of extracellular matrix (ECM), including ECM harvested from tissue donors, are used to preserve the unique characteristics of cultured cells. Using this method, cells cultured from the annulus fibrosis are more likely to retain the unique features of fibrocytes, and cells of the annulus fibrosis, if portions of annulus fibrosis ECM are included in the culture media for culturing annulus fibrosis cells. The technique is extendable to other types of cell and tissue cultures, including chondrocytes, nucleus pulposis cells, and so forth. Additional therapeutic substances may be added cell/matrix culture In addition, although the cultured cells are well suited to annulus fibrosis augmentation and/or transplantation, the invention is not limited to treatment of the intervertebral disc. For example, the invention could also be used to treat other tissues of the body such as the meniscus of the knee. The process may also be used to repair or replace other tissues or organs of the body such as the pancreas, liver, kidney, heart, etc.

### REFERENCE TO RELATED APPLICATIONS

This is a continuation-in-part of U.S. patent application Ser. No. 09/688,716, filed Oct. 16, 2000, which is a continuation-in-part of U.S. patent application Ser. No. 09/638,726, now U.S. Pat. No. 6,340,369 and U.S. patent application Ser. No. 09/415,382, now U.S. Pat. No. 6,419,704. The entire content of each application is incorporated herein by reference.

### FIELD OF THE INVENTION

This invention relates generally to cell and tissue culture and, in particular, to the use of the extracellular matrix (ECM) from appropriate donors to preserve the unique characteristics of cultured biologic materials.

### BACKGROUND OF THE INVENTION

Intervertebral discs provide mobility and a cushion between the vertebrae. At the center of the disc is the nucleus pulposus. The nucleus pulposus is surrounded by the annulus fibrosis, which is comprised of cells (fibrocyte-like and chondrocyte-like), collagen fibers, and non-fibrillar extracellular matrix.

Although transplantation of living cells risks rejection by graft host reaction, my co-pending U.S. patent application Ser. No. 09/688,716 broadly recognizes that transplantation of the extracellular matrix is unlikely to incite graft host reaction. In the preferred embodiment, fibrocytes are harvested, cultured, then added to annulus fibrosis extracellular matrix obtained from a recently deceased human, animal, or other suitable donor. The combined annulus fibrosis is then introduced into the injured or diseased disc.

The method may further include the step of adding one or more therapeutic substances to the cells or annular tissue prior to transplantation. Such therapeutic substances could include culture media, growth factors, differentiation factors, hydrogels, polymers, antibiotics, anti-inflammatory medications, immuno-suppressive medications, or any useful combination thereof.

It is known, however, that cultured cells, particularly cultured human cells, loose their phenotype, or specific cell characteristics after several generations. That is, fibrocytes, chondrocytes, nucleus pulposis cells, and other cells gradually lose their unique features as the cells reproduce under laboratory conditions. Each successive generation of cultured cells become more like a generic cell. Thus, any technique for preserving cell-specific attributes in culture would be of benefit to the medical community.

### SUMMARY OF THE INVENTION

According to this invention, pieces of extracellular matrix (ECM), including ECM harvested from tissue donors, are used to preserve the unique characteristics of cultured cells. Using this method, cells cultured from the annulus fibrosis are more likely to retain the unique features of fibrocytes, and cells of the annulus fibrosis, if portions of annulus fibrosis ECM are included in the culture media for culturing annulus fibrosis cells. The technique is extendable to other types of cell and tissue cultures, including chondrocytes, nucleus pulposis cells, and so forth.

Additional therapeutic substances may be added cell/matrix culture. For example, resorbable culture medium, tissue growth or differentiation factors (recombinant generated morphogenetic proteins, PDGF, TGF-β, EGF/TGF-α, IGF-I, βFGF), hydrogels, absorbable or nonresorbable synthetic or natural polymers (collagen, fibrin, polyglycolic acid, polylactic acid, polytetrafluoroethylene, etc.), antibiotics, anti-inflammatory medication, immunosuppressive medications, etc. may be used.

Although the cultured cells are well suited to annulus fibrosis augmentation and/or transplantation, the invention is not limited to treatment of the intervertebral disc. For example, the invention could also be used to treat other tissues of the body such as the meniscus of the knee. The process may also be used to repair or replace other tissues or organs of the body such as the pancreas, liver, kidney, heart, etc. Healthy live cells would be obtained thorough biopsy and tissue culture. The live cells would be added to the extracellular matrix of tissues or organs harvested to recently deceased human or animals to preserve their respective phenotype.

### DETAILED DESCRIPTION OF THE INVENTION

Broadly according to the invention, annulus fibrosis extracellular matrix material is added to cultured cells to help preserve cell identity as the cells reproduce under laboratory conditions, including reproduction over successive generations. The approach is applicable to various cells and tissues, including fibrocytes, chondrocytes, nucleus pulposis cells, etc. The cultured cells may then be added to, then sewn or otherwise placed relative to an injured or diseased disc or other area of the body, as discussed in further detail below.

The cells to be cultured and extracellular matrix are preferably harvested from a live human, though recently deceased human or animal donors may alternatively be used. Depending upon the extent of the harvest, the recipient may function at least in part as a donor, or the tissues from others, including fetal sources, may be used, preferably having a familial relationship to minimize or avoid the need for immunosuppressive substances. Guidelines for tissue procurement including surgical technique of removal, number of hours between death of the donor and tissue procurement, and testing of the donor for infectious disease, are well described.

Following annulus fibrosis harvest, the tissue is processed to kill the living cells. Care is taken to preserve the extracellular matrix. Guidelines for processing the harvested annulus fibrosis as described are well known to those skilled in the art. For example, the tissue could be frozen and thawed.

Fibrocytes may be obtained from a tendon of the patient. For example, a palmaris longus tendon could be removed from one arm of the patient. But for the addition of the ECM material, the harvested fibrocytes are isolated and cultured using standard techniques, as disclosed in my co-pending U.S. patent application Ser. No. 09/688,716. In particular, the harvested cells may be grown in Hamm's F-12 culture media, 10% fetal calf serum, L-glutamine (292.mu.g/cc), penicillin (100 u/cc), streptomycin (100.mu.g/cc), and asorbic acid (5.mu.g/cc) at 37° C. The above method is described in U.S. Pat. No. 6,060,053, which is incorporated in its entirety herein by reference.

Precursor cells of the annulus fibrosis, annulus fibrosis cells, chondrocytes, nucleus pulposis cells, or other living cells may also be used. The living cells and extracellular matrix may be added to the patient's disc immediately after combination or after a period of time to allow attachment of the cells and matrix.

Additional therapeutic substances may be added cell/matrix culture. For example, resorbable culture medium, tissue growth or differentiation factors (recombinant generated morphogenetic proteins, PDGF, TGF-β, EGF/TGF-α, IGF-I, βFGF), hydrogels, absorbable or nonresorbable synthetic or natural polymers (collagen, fibrin, polyglycolic acid, polylactic acid, polytetrafluoroethylene, etc.), antibiotics, anti-inflammatory medication, immunosuppressive medications, etc. may be used.

Although the cultured cells are well suited to annulus fibrosis augmentation and/or transplantation, the invention is not limited to treatment of the intervertebral disc. For example, the invention could also be used to treat other tissues of the body such as the meniscus of the knee. The process may also be used to repair or replace other tissues or organs of the body such as the pancreas, liver, kidney, heart, etc. Healthy live cells would be obtained thorough biopsy and tissue culture. The live cells would be added to the extracellular matrix of tissues or organs harvested to recently deceased human or animals to preserve their respective phenotype.

I claim:

1. A method of preserving the phenotype of a cultured cell, comprising the steps of:
harvesting cells to be cultured from a suitable donor;
harvesting the extracellular matrix (ECM) of the annulus fibrosis from a living or recently deceased human or animal; and
culturing the cells along with portions of the ECM to preserve the phenotype of the cultured cell.

2. The method of claim 1, wherein the cells to be cultured are fibrocytes, chondrocytes, or nucleus pulposis cells.

3. The method of claim 1, further including the step of transplanting the cultured cells into or onto a vertebral disc.

4. The method of claim 1, further including the step of adding one or more therapeutic substances to the cell culture.

5. The method of claim 4, wherein the therapeutic substances include one or more of the following:
culture media, growth factors, differentiation factors, hydrogels, polymers, antibiotics, anti-inflammatory medications, or immunosuppressive medications.
